(19) United States
(12) Patent Application Publication
(10) Pub. No.: US 2016/0235448 A1
(43) Pub. Date: Aug. 18, 2016

(54) SPINAL PLATE SELECTION AND POSITIONING SYSTEM

(71) Applicant: Retrospine Pty Ltd, Sydney (AU)

(72) Inventor: Kevin Seex, Sydney (AU)

(21) Appl. No.: 14/295,156

(22) Filed: Jun. 3, 2014

Related U.S. Application Data
(63) Continuation of application No. 14/157,412, filed on Jan. 16, 2014, now abandoned.
(60) Provisional application No. 61/753,420, filed on Jan. 16, 2013.

(51) Int. Cl. A61B 17/70; A61F 2/44

(52) U.S. Cl. CPC A61B 17/7059; A61F 2/4455

(57) ABSTRACT

A spinal plate selection and positioning system is provided for use in a spinal fusion procedure. The system may comprise an elongated guide member that is removably securable to an interbody cage by a holding rod. An interbody plate may be aligned and positioned above the interbody cage installed in a disc space. A drill guide may also be aligned and positioned above the interbody plate. The drill guide may be utilized to drill pilot holes in the vertebrae defining the disc space. Fasteners to secure the interbody plate may also by installed using the drill guide. The use of the guide member ensures that the interbody plate is properly aligned and positioned with respect to the interbody cage.

### CROSS-REFERENCE TO RELATED APPLICATIONS

This application is a continuation of U.S. patent application Ser. No. 14/157,412, filed Jan. 16, 2014, which claims the benefit of U.S. Provisional Application No. 61/753,420, filed Jan. 16, 2013, entitled “SPINAL PLATE SELECTION AND POSITIONING SYSTEM,” which is hereby incorporated by reference herein in its entirety, including but not limited to those portions that specifically appear hereinafter, the incorporation by reference being made with the following exception: In the event that any portion of the above-referenced applications is inconsistent with this application, this application supercedes said above-referenced applications.

### STATEMENT REGARDING FEDERALLY SPONSORED RESEARCH OR DEVELOPMENT

Not Applicable.

### BACKGROUND

1. The Field of the Present Disclosure

The present disclosure relates generally to medical devices, and more particularly, but not necessarily entirely, to medical implants and instrumentation utilized in spinal-fusion surgical procedures.

2. Description of Related Art

Spinal fusion surgery is often performed to reduce pain caused by aging or injury. Spinal fusion surgery typically involves fusing two or more vertebrae in the spinal column. Fusion of the vertebrae may be accomplished using a bone fixation device, such as an interbody plate. In particular, rigid interbody plates may be secured to the vertebrae. The interbody plates may stabilize the spinal column. Fasteners, such as bone screws, may be utilized to secure the interbody plates to the vertebrae. For this reason, interbody plates are generally rigid but may have some adjustability for sizing purposes. Rods may sometimes also be utilized to provide greater stabilization.

It is further known to use an interbody cage in conjunction with a spinal fusion procedure. In particular, an interbody cage may be implanted by press fit into the disc space between two adjacent vertebrae. (This, of course, may first require removal of the patient's natural disc.) In this regard, the use of an interbody cage has been found to reduce post-operative discomfort and pain. Interbody cages may take a wide variety of forms. For example, interbody cages may be formed from alloys or plastic. In some instances, interbody cages are packed with autologous bone material in order to promote fusion. That is, this supplementary bone material, such as an allograft or an autograft, may be used in conjunction with the patient's natural bone growth process to fuse the vertebrae. In other instances, interbody cages may be made entirely of, or partly of, either a human graft (allograft or autograft) or an animal graft (xenograft).

An interbody plate and an interbody cage may be utilized together. In this instance, the interbody plate may extend over a disc space containing the interbody cage. The interbody plate may then be secured to the two vertebrae defining the disc space using fasteners. An interbody plate is ideally positioned parallel to the longitudinal axis of the spine and symmetrically over the disc space. In practice, however, proper alignment and positioning of the interbody cage may be difficult to accomplish due to (i) narrow wounds which obscure surgical landmarks; (ii) blood or other tissue may obscure landmarks; (iii) the interbody plate itself may obscure landmarks; (iv) drill guides that are used to drill pilot holes may further obscure landmarks; and (v) the interbody plate may move prior to being secured without the surgeon being aware.

In the past, some techniques have been developed in an attempt to properly align and position interbody plates and interbody cages. One technique includes the use of a trial cage connected to a drill guide. In particular, the trial cage is temporarily installed into the disc space. The attached drill guide is then utilized to drill pilot holes in the adjacent vertebrae. The trial cage and drill guide are then removed and the interbody cage is implanted into the disc space. The plate is then positioned and fastened to the vertebrae using the pilot holes. This technique, however, has limitations. One limitation is that there is no assurance that the trial cage and the actual interbody cage are located in the same position. Another limitation is that there is no assurance that the interbody plate is positioned correctly with respect to the pilot holes. A further limitation is that the positioning of the interbody cage relative to the interbody plate is not controlled.

Another attempt to properly align and position interbody plates and interbody cages is taught by Fraser et al. (U.S. Patent Publication No. 2011/004253). Fraser teaches the use of an interbody plate that includes integral mating elements that are adapted to slidably engage an interbody cage. One draw back to Fraser's teachings is that there is no assurance that the interbody plate is centered over the interbody cage or that the cage is properly positioned in the disc space prior to the plate being secured.

Despite the advantages of known alignment and positioning techniques, improvements are still being sought. For example, many of the prior art devices cannot assure that an interbody plate and interbody cage are ideally positioned. The prior art is thus characterized by several disadvantages that are addressed by the present disclosure. The present disclosure minimizes, and in some aspects eliminates, the above-mentioned failures, and other problems, by utilizing the methods and structural features described herein.

The features and advantages of the present disclosure will be set forth in the description which follows, and in part will be apparent from the description, or may be learned by the practice of the present disclosure without undue experimentation. The features and advantages of the present disclosure may be realized and obtained by means of the instruments and combinations particularly pointed out in the appended claims.

### BRIEF DESCRIPTION OF THE DRAWINGS

The features and advantages of the disclosure will become apparent from a consideration of the subsequent detailed description presented in connection with the accompanying drawings in which:

FIG. 1 is a perspective view of an interbody cage according to an embodiment of the present disclosure;

FIG. 2 is a perspective view of a holding rod according to an embodiment of the present disclosure;

FIG. 3 is a perspective view of an interbody plate according to an embodiment of the present disclosure;

FIG. 4 is a perspective view of a guide member according to an embodiment of the present disclosure;

FIG. 5 is a perspective view of a drill guide according to an embodiment of the present disclosure;

FIG. 6 is another perspective view of the drill guide shown in FIG. 5 according to an embodiment of the present disclosure;

FIG. 7 is a perspective view of another drill according to an embodiment of the present disclosure;

FIG. 8 is an exploded view of an assembly according to an embodiment of the present disclosure;

FIG. 9 is a partially exploded view of the assembly shown in FIG. 8 according to an embodiment of the present disclosure;

FIG. 10 depicts a procedure for installing an interbody cage in a disc space according to an embodiment of the present disclosure;

FIG. 11 depicts a procedure for drilling pilot holes and installing plate fasteners in a vertebra according to an embodiment of the present disclosure;

FIG. 12 depicts a procedure for drilling pilot holes and installing plate fasteners in a vertebra according to an embodiment of the present disclosure;

FIG. 13 depicts a procedure for removing a drill guide according to an embodiment of the present disclosure;

FIG. 14 depicts an installed interbody plate and interbody cage according to an embodiment of the present disclosure; and

FIG. 15 depicts a pair of installed interbody plates and interbody cages according to an embodiment of the present disclosure.

### DETAILED DESCRIPTION

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

In describing and claiming the present disclosure, the following terminology will be used in accordance with the definitions set out below. It must be noted that, as used in this specification and the appended claims, the singular forms “a,” “an,” and “the” include plural referents unless the context clearly dictates otherwise. As used herein, the terms “comprising,” “including,” “containing,” “characterized by,” and grammatical equivalents thereof are inclusive or open-ended terms that do not exclude additional, unrecited elements or method steps.

Applicant has discovered a spinal plate selection and positioning system for use in a spinal fusion surgery. In an embodiment, the system may include a guide member removably attached to an interbody cage by a holding rod. After the interbody cage has been installed into a disc space defined between a first vertebra and a second vertebra using the holding rod, the guide member may be utilized to align and position an interbody plate over the disc space. The guide member may center the interbody plate over the interbody cage and also prevent the interbody plate from rotating about a longitudinal axis of the guide member. Thus, the guide member may align and position the interbody plate with reference to the interbody cage since the guide member and interbody cage are coupled.

A drill guide may then also be aligned and positioned over the disc space using the guide member. Pilot holes may then be drilled in the vertebrae defining the disc space using the drill guide. Using the drill guide, fasteners may be installed to secure the interbody plate to the vertebrae. The drill guide may then be removed from the guide member and the guide member may be detached from the interbody cage, now secured in the disc space beneath the interbody plate.

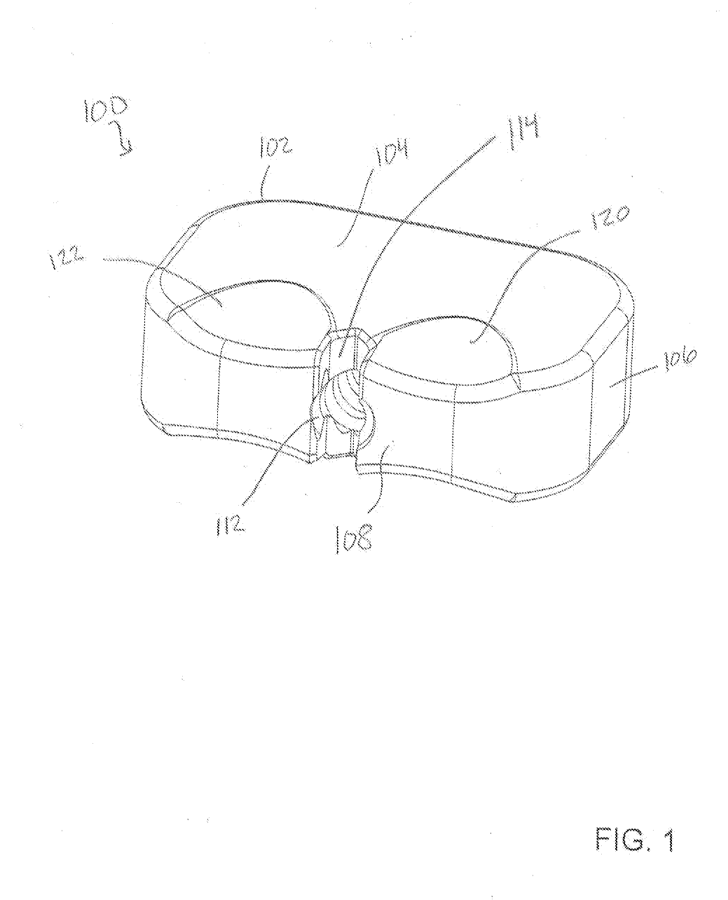

Referring now to FIG. 1, there is depicted an exemplary interbody cage 100 pursuant to an embodiment of the present disclosure. The interbody cage 100 may include a body member 102. The body member 102 may have a superior surface 104 and an inferior surface 105 (not explicitly visible in FIG. 1, but understood to be opposite of the superior surface 104). A sidewall 106 may circumscribe the body member 102. The sidewall 106 may extend between the superior surface 104 and the inferior surface 105. The sidewall 106 may define, for the body member 102, a proximal surface 108 and a distal surface 110 (not explicitly visible in FIG. 1, but understood to be opposite of the proximal surface 108).

A threaded bore 112 may be formed in the proximal surface 108 of the sidewall 106. As will be explained in more detail hereinafter, the threaded bore 112 may engage a threaded end of a holding rod that is utilized to position and implant the interbody cage 100 in a disc space defined between two adjacent vertebrae. A key slot 114 may also be formed in the proximal surface 108 of the sidewall 106. The key slot 114 may extend from the superior surface 104 to the inferior surface 105 of the body member 102. In an embodiment, the key slot 114 may pass through the threaded bore 112. As will be explained in more detail hereinafter, the key slot 114 may engage key members extending from a guide member.

Formed in the superior surface 104 of the body member 102 may be grooves or cutouts 120 and 122. As will be explained in detail hereinafter, the grooves 120 and 122 may form reliefs in the superior surface 104 to allow passage of fasteners utilized to secure an interbody plate to vertebrae in a spinal column. The grooves 120 and 122 may extend from the proximal surface 108 of the sidewall 106 toward the distal surface 110 of the sidewall 106. The grooves 120 and 122 may taper upwards, in the superior direction, as they extend toward the distal surface 110 such that they taper out.

One of the grooves 120 and 122 may be disposed on either side of the threaded bore 112. It will be appreciated that the inferior surface 105 of the body member may contain grooves similar in size, shape and location to the grooves 120 and 122 in the superior surface 104. In an embodiment, the grooves in the inferior surface 105 may differ in size, shape, and location to the grooves 120 and 122 in the superior surface 104.

Although the body member 102 is depicted as a solid member, it will be appreciated that the body member 102 may take a wide variety of configurations according to embodiments of the present disclosure. In an embodiment, the body member 102 may comprise a hollow interior portion for receiving a bone graft. Further, the body member 102 may be formed from a wide range of biocompatible materials as is known to one having ordinary skill.

Pursuant to an embodiment of the present disclosure, the present disclosure may provide a plurality of interbody cages of varying height, i.e., the distance between the superior surface 104 and the inferior surface 105, to accommodate disc spaces of varying sizes. In an embodiment, a surgeon may trial fit multiple interbody cages until the right fit is obtained.

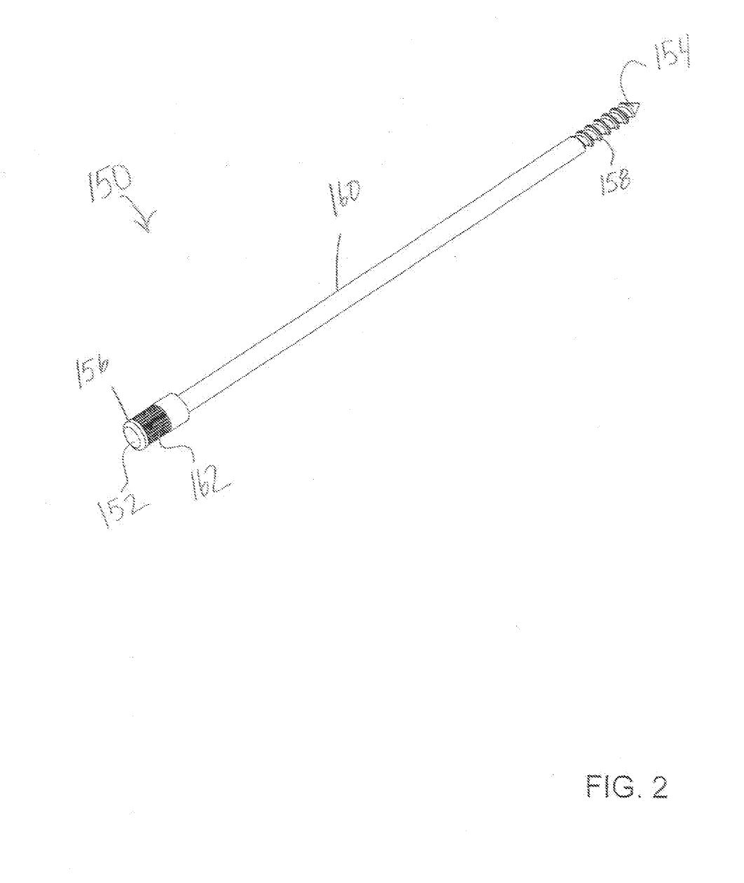

Referring now to FIG. 2, there is depicted a holding rod 150 pursuant to an embodiment of the present disclosure. The holding rod 150 may extend from a proximal end 152 to a distal end 154. A knob 156 may be located at the proximal end 152 of the holding rod 150. The distal end 154 may include a plurality of threads 158. A shaft 160 may extend between the proximal end 152 and the distal end 154. The knob 156 may include grip enhancing means 162, such as serrations. In an embodiment, the threads 158 on the distal end 154 of the holding rod 150 are configured and adapted to engage the threaded bore 112 of the interbody cage 100 (see FIG. 1). It will be appreciated that the holding rod 150 may be utilized to facilitate the installation of the interbody cage 100 into a disc space between two adjacent vertebrae.

The present disclosure further contemplates other means of securing the holding rod 150 to the interbody cage 100. In an embodiment, the holding rod 150 may engage the interbody cage 100 by a snap or press fit. In an embodiment, the holding rod 150 may include an expandable head, activated proximally, that engages a receiving portion in the interbody cage 100 when the head is expanded. In an embodiment, the holding rod 150 may engage the interbody cage 100 using a twist-lock mechanism.

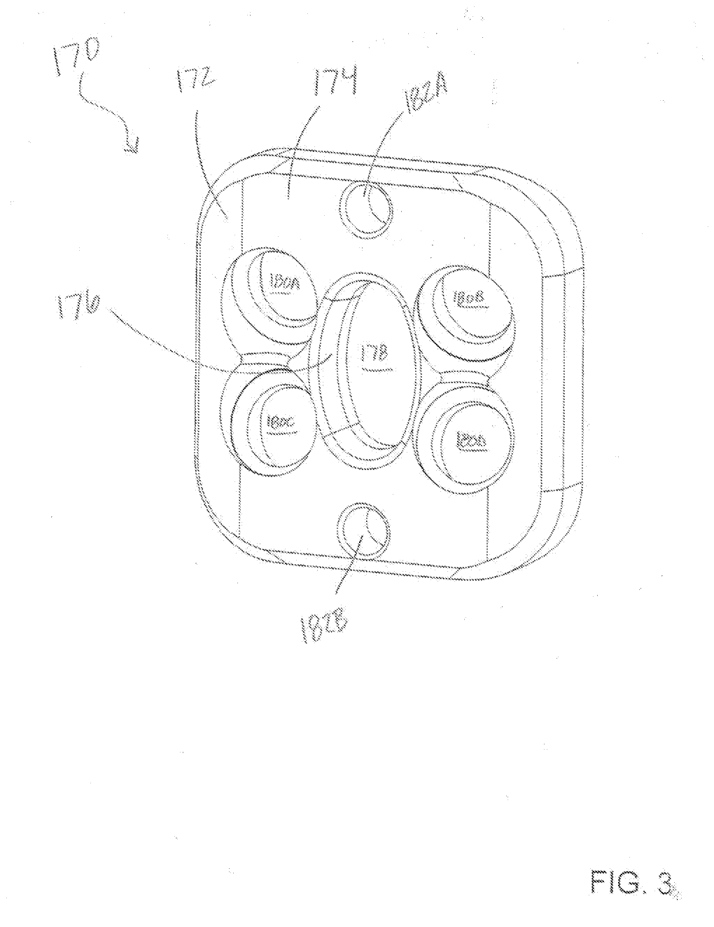

Referring now to FIG. 3, there is depicted an interbody plate 170 pursuant to an embodiment of the present disclosure. The plate 170 may include a body member 172 according to an embodiment of the present disclosure. The body member 172 may include a proximal end 174 and a distal end 175 (not explicitly visible in FIG. 3, but the distal end 175 is understood to be on the opposite side of the interbody plate 170 from the proximal end 174). In an embodiment, the body member 172 of the plate 170 is substantially rectangular in shape.

An inner surface 176 of the body member 172 may define a guide hole 178. In an embodiment, the guide hole 178 may be located in the center of the body member 172. The guide hole 178 may extend from the proximal end 174 to the distal end 175. In an embodiment, the guide hole 178 may be noncircular. In an embodiment, the guide hole 178 may be oval.

The body member 172 may further comprise fastener holes 180A, 180B, 180C and 180D. Holes 180A and 180B may define a first set of holes and holes 180C and 180D may define a second set of holes. The first set of holes 180A, 180B may allow fasteners (not explicitly shown) to secure the plate 170 to a first vertebra (not explicitly shown) and the second set of holes 180C, 180D may allow fasteners (not explicitly shown) to secure the plate 170 to a second vertebra (not explicitly shown). One of the first set of holes 180A, 180B may be disposed on either side of the guide hole 178. One of the second set of holes 180C, 180D may be disposed on either side of the guide hole 178. The first set of holes 180A, 180B may be angled. The second set of holes 180C, 180D may be angled.

The body member 172 may further comprise a pair of guide holes 182A and 182B. The guide holes 182A and 182B may facilitate the positioning of a drill guide over the holes 180A-180D as will be explained in detail hereinafter.

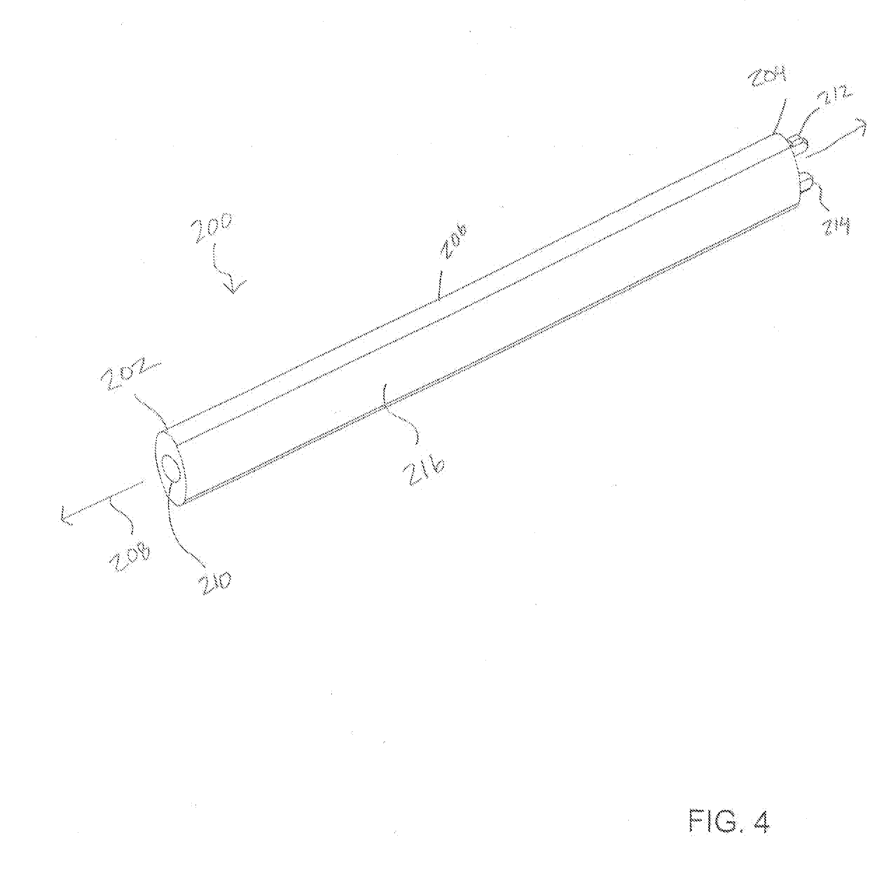

Referring now to FIG. 4, there is depicted an elongated guide member 200 pursuant to an embodiment of the present disclosure. The guide member 200 may have a proximal end 202 and a distal end 204. A body portion 206 of the guide member 200 may extend between the proximal end 202 and the distal end 204. The body portion 206 may extend along a longitudinal axis 208. A cannulation 210 may extend from the proximal end 202 to the distal end 204.

In an embodiment, the guide member 200 may include key members 212 and 214 extending from the distal end 204. The key members 212 and 214 are configured and adapted to engage the key slot 114 of the interbody cage 100 (see FIG. 1) as will be explained in more detail hereinafter. It will be appreciated that the use of the key members 212 and 214, and the key slot 114, prevent the elongated guide member 200 from rotating about its longitudinal axis 208 when removably secured to the interbody cage 100.

In an embodiment, the cannulation 210 is configured and adapted to allow the shaft 160 of the holder rod 150 (see FIG. 2) to be inserted into, and pass through, the guide member 200. In an embodiment, a cross section of the guide member 200 in a plane perpendicular to its longitudinal axis 208 may be noncircular. In an embodiment, the cross section is oval. The guide member 200 may have an outer surface 216. In an embodiment, the holding rod 150 and the guide member 200 may be formed as a single unit rather than as a two piece assembly as shown.

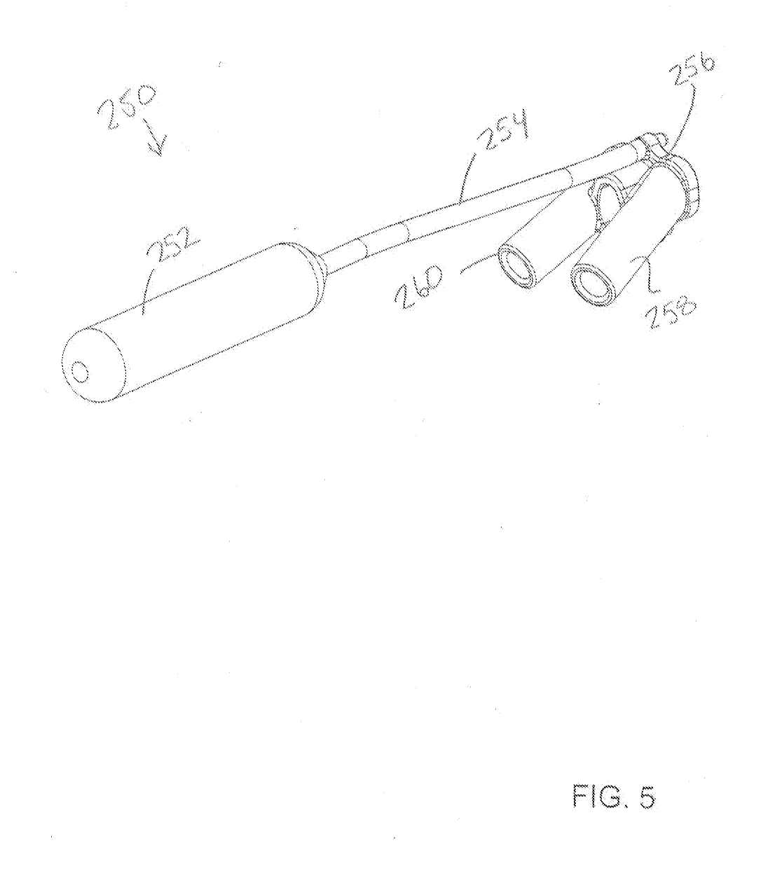

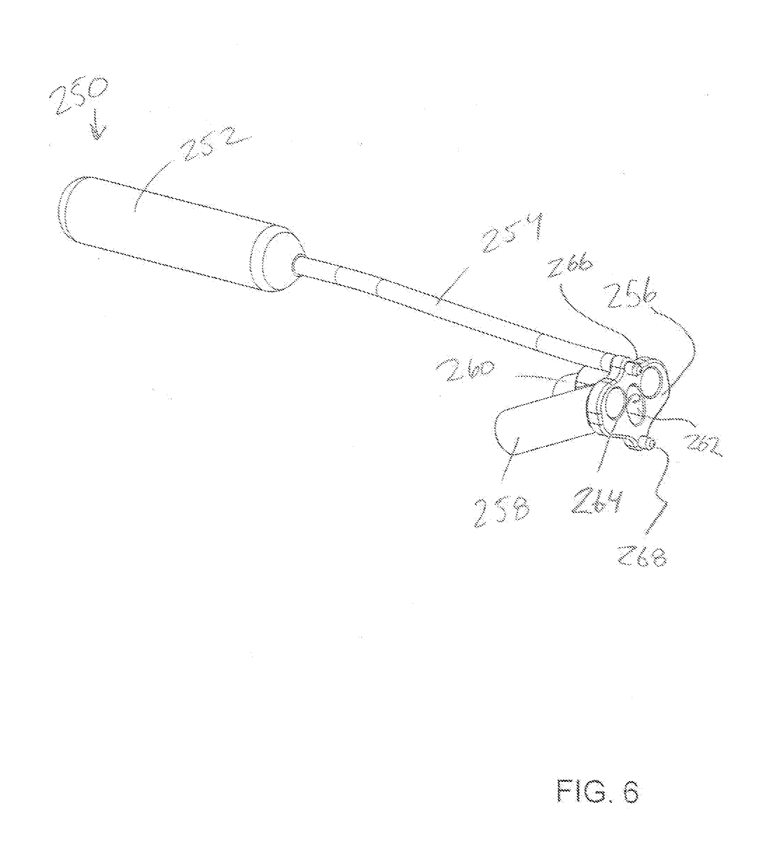

Referring now to FIGS. 5 and 6, there is depicted a drill guide 250 pursuant to an embodiment of the present disclosure. The drill guide 250 may comprise a handle 252. A shaft 254 may extend from the handle 252. A base member 256 may be attached to the distal end of the shaft 254. A first guide tube 258 and a second guide tube 260 may extend from the base member 256. It will be appreciated that the first guide tube 258 and the second guide tube 260 may each include a hollow passageway for allowing the passage of a drill bit and fasteners through the base member 256. Further, while the drill guide 250 is shown with two guide tubes, it will be appreciated that, pursuant to embodiments of the present disclosure, the drill guide 250 may comprise a single guide tube or any number of guide tubes.

As perhaps best seen in FIG. 6, an inner surface 262 of the base member 256 may define a guide hole 264. In an embodiment, the guide hole 264 is noncircular. In an embodiment, the guide hole 264 is oval. As will be explained in further detail hereinafter, the guide hole 264 may receive the guide member 200 to align and position the drill guide 250 over a disc space. Further, extending from the base member 256 may be a first guide post 266 and a second guide post 268. As will be explained in more detail hereinafter, the first guide post 266 and the second guide post 268 may align the drill guide 250 with respect to the interbody plate 170.

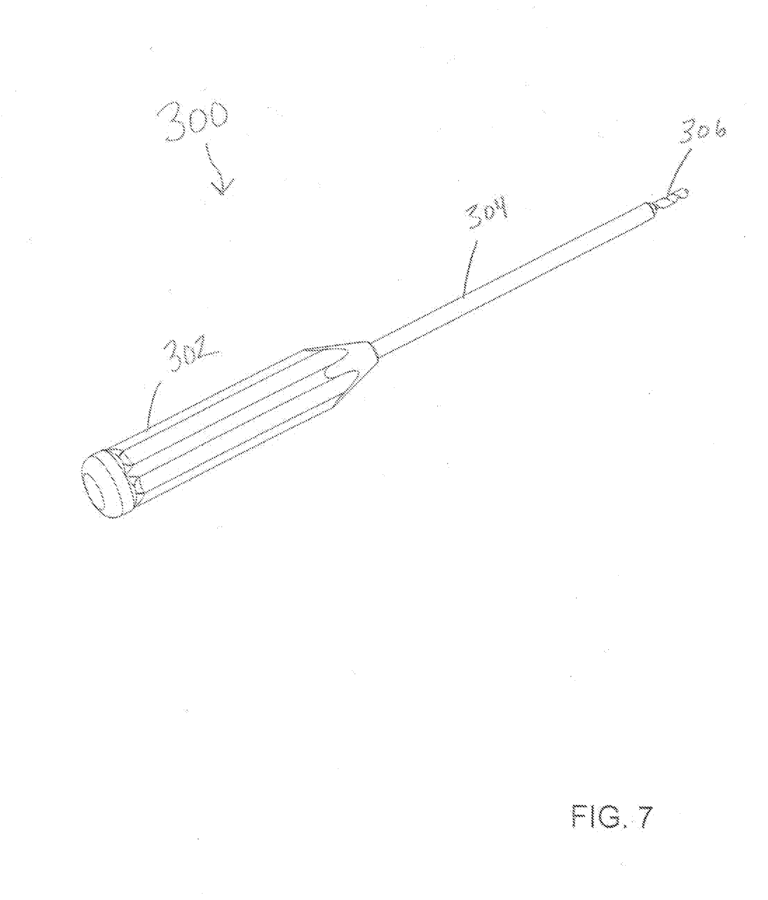

Referring now to FIG. 7, there is depicted a surgical drill 300 according to an embodiment of the present disclosure. The drill 300 may be a manually operated drill. In an embodiment, the drill 300 may be a powered surgical drill.

The drill 300 may comprise a handle 302. A shaft 304 may extend from the handle. It will be appreciated that the diameter of the shaft 304 may allow the shaft 304 to snugly fit in the passageways in the hollow tube guides 258 and 260 of the drill guide 250. Disposed on the end of the shaft 304 may be a drill bit 306. The drill bit 306 may be utilized to drill pilot holes in vertebrae as is known to one having ordinary skill.

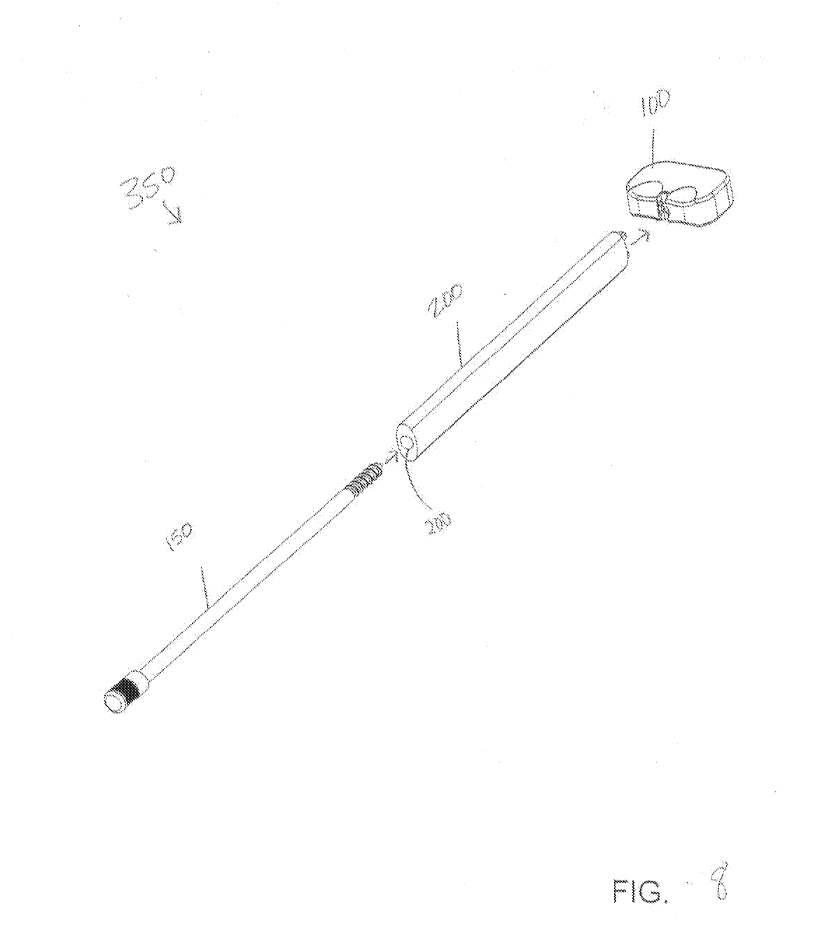

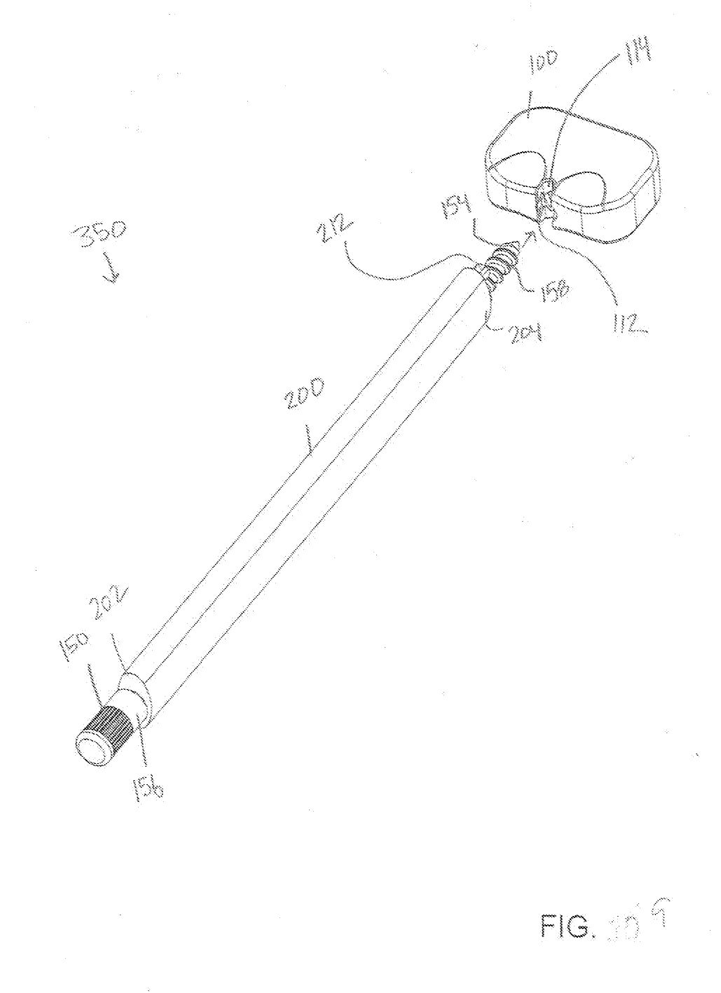

Referring now to FIGS. 8, 9, and 10, there is depicted an exploded view of a system 350 for spinal fixation. The system 350 may comprise the interbody cage 100, the guide member 200, and the holding rod 150. As perhaps best viewed in FIG. 9, the holding rod 150 is installed into the cannulation 210 of the guide member 200. The proximal end 202 of the guide member 200 may abut against the knob 156 of the holding rod 150. The length of the holding rod 150 may be sufficient such that the threads 158 on the distal end 154 extend from the distal end 204 of the guide member 200.

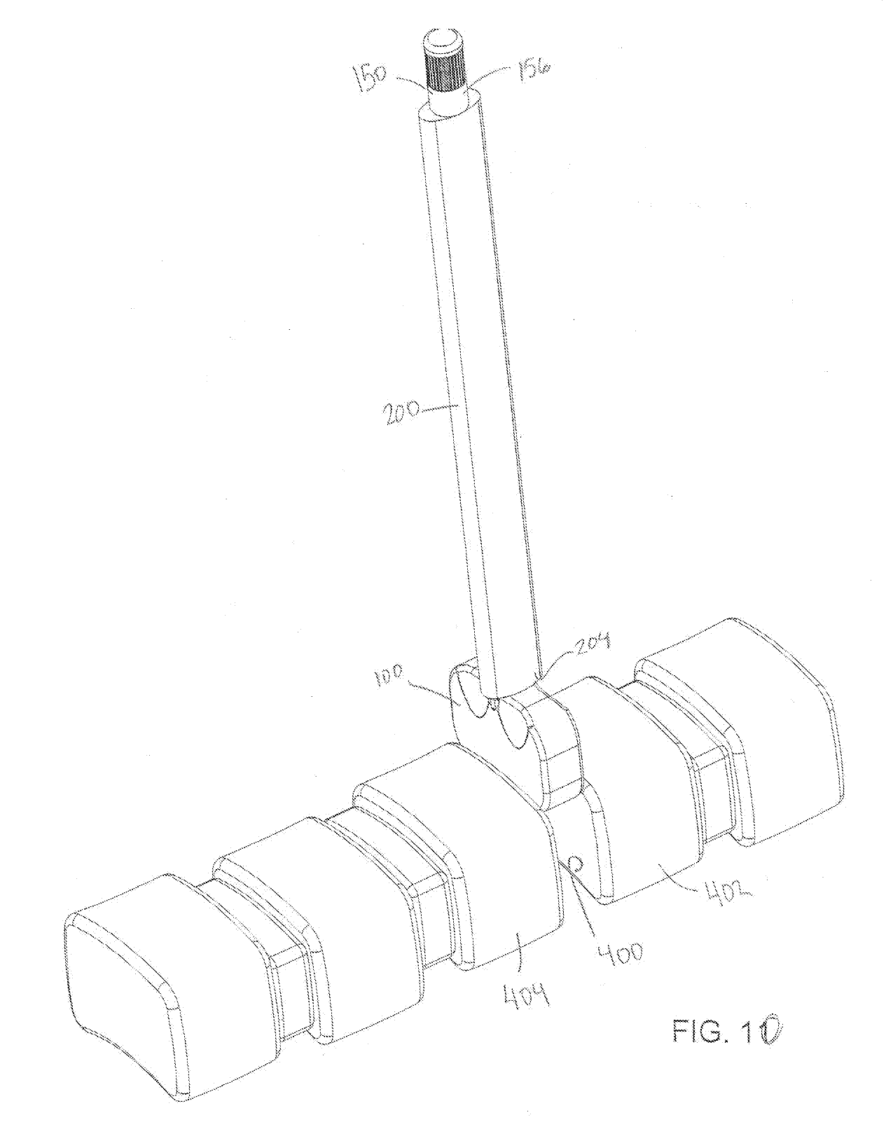

As perhaps best observed in FIG. 10, the distal end 154 of the holding rod 150 is installed into the threaded bore 112 of the interbody cage 100. The knob 156 may provide a grip for a surgeon to grasp and position the interbody cage 100. In addition, the knob 156 may receive hits from an impaction tool, such as a hammer, to impact the interbody cage 100. The distal end 204 of the guide member 200 may serve as a depth gauge when installing the interbody cage 100 by abutting against two vertebrae defining the disc space.

Further, the key members 212 and 214 extending from the distal end 204 of the guide member 200 may engage the key slot 114 of the interbody cage 100. It will be appreciated by those of ordinary skill that the key slot engagement positively locates the guide member 200 with respect to the interbody cage 100 and prevents the guide member 200 from moving or rotating about the holding rod 150 or its longitudinal axis 208.

Referring now to FIGS. 10-14, there is depicted a spinal fusion procedure according to an embodiment of the present disclosure. As previously described, the holding rod 150 may be utilized to removably secure the guide member 200 to the interbody cage 100 as observed in FIG. 10. This may be done prior to installing the interbody cage 100 into the disc space.

A disc space 400 defined between a first vertebra 402 and a second vertebra 404 may be prepared by removing the natural disc as is known to those having ordinary skill. Next, the interbody cage 100 may be positioned into the disc space 400 by a surgeon using the holding rod 150 having the guide member 200 disposed thereon. The interbody cage 100 may be impacted into the disc space 400. Once the interbody cage 100 has been installed in the disc space 400, it may be positioned using the holding rod 150. The depth of the cage 100 in the disc space may be determined by the distal end 204 of the guide member 200, which may abut against the first vertebra 402 and the second vertebra 404. It will be appreciated that the guide member 200 may extend outside of the body of the patient when attached to the interbody cage 100.

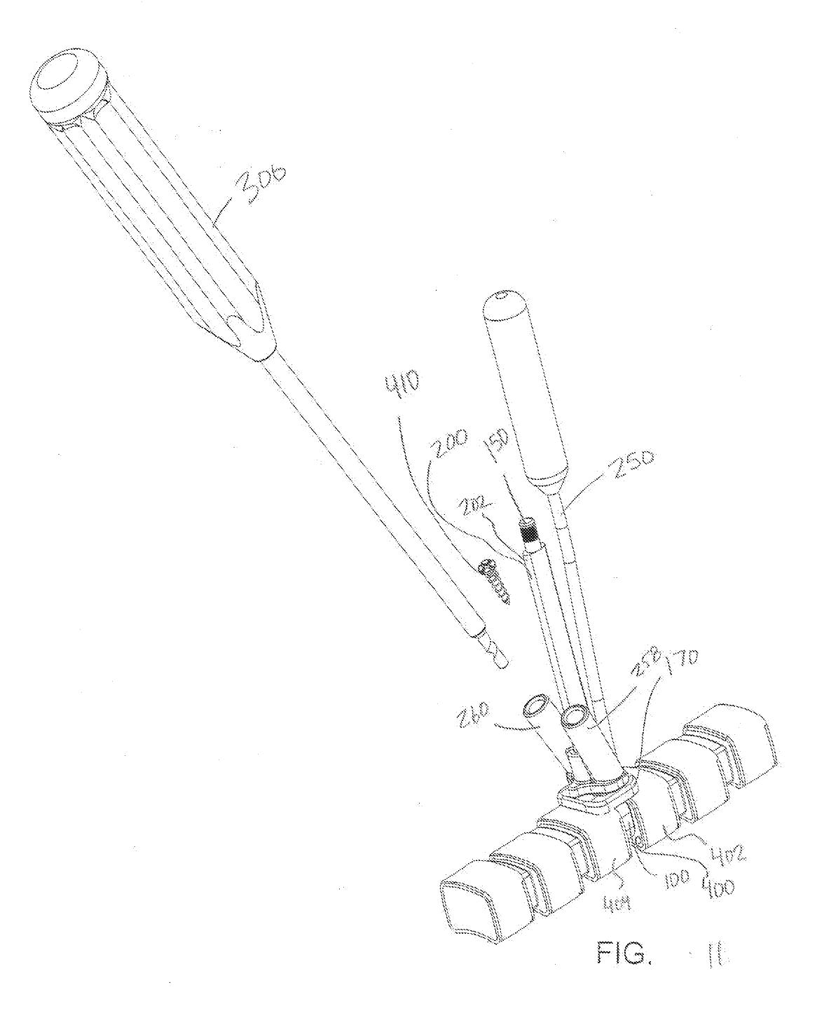

As seen in FIG. 11, with the interbody cage 100 positioned in the disc space 400, the interbody plate 170 is aligned and positioned over the disc space 400 using the guide member 200. In particular, the proximal end 202 of the guide member 200 is installed into the guide hole 178 of the interbody plate 170. The plate 170 is then slid down the length of guide member 200 into position over the disc space 400.

The guide member 200 may position and align the interbody plate 170 above the interbody cage 100. In particular, the inner surface 176 of the plate 170 that defines the guide hole 178 may engage the outer surface 216 of the guide member 200. It will be appreciated that the noncircular nature of the cross section of the guide member 200 prevents the plate 170 from rotating or otherwise becoming misaligned over the disc space 400.

Once the interbody plate 170 is positioned over the disc space 400, the drill guide 250 may be installed onto the guide member 200 and positioned over the interbody plate 170. In particular, the guide hole 264 of the drill guide 250 is installed onto the proximal end 202 of guide member 200 and slid into position over the interbody plate 170. At this point, the first guide post 266 and the second guide post 268 of the base member 256 of the drill guide 250 may engage the pair of guide holes 182A and 182B of the interbody plate 170 to ensure that the drill guide 250 is properly positioned with respect to the plate 170 and the cage 100.

In an embodiment, the drill guide 250 and the plate 170 may be preassembled such that they can be installed as one unit over the guide member 200. Various methods of securing the drill guide 250 and the plate 170 together are envisioned and fall within the scope of the present disclosure. In an embodiment, the guide posts 266 and 268 may have a press or snap fit into the guide holes 182A and 182B of the plate 170. In an embodiment, the guide posts 266 and 268 may threadably engage the guide holes 182A and 182B of the plate 170. In an embodiment, the drill guide 250 and the plate 170 may be magnetically coupled. Thus, the present disclosure contemplates various engagements means to secure the drill guide 250 and the plate 170 together such that they can be inserted together. Once the fasteners are installed to secure the plate 170, as explained below, the drill guide 250 may be detached from the plate 170 to allow the drill guide 250 to be removed.

Once the drill guide 250 is in place and locked with the plate 170, the drill 300 may be utilized to drill pilot holes in the vertebrae 402 and 404. In particular, with the guide tubes 258 and 260 of the drill guide 250 aligned over the first set of holes 180A, 180B, the drill 300 may be utilized to drill two pilot holes in the vertebra 402. It will be appreciated that the drill bit 306 passes through the holes 180A and 180B in the plate 170. Further, as the diameter of the shaft 304 of the drill 300 is just slightly smaller than the diameter of the guide tubes 258 and 260 of the drill guide 250, the bit 306 will be properly aligned. Once the pilot holes have been created, and before the drill guide 250 is removed, fasteners 410 may be installed into the guide tubes 258 and 260 to secure the plate 170 to the first vertebra 402.

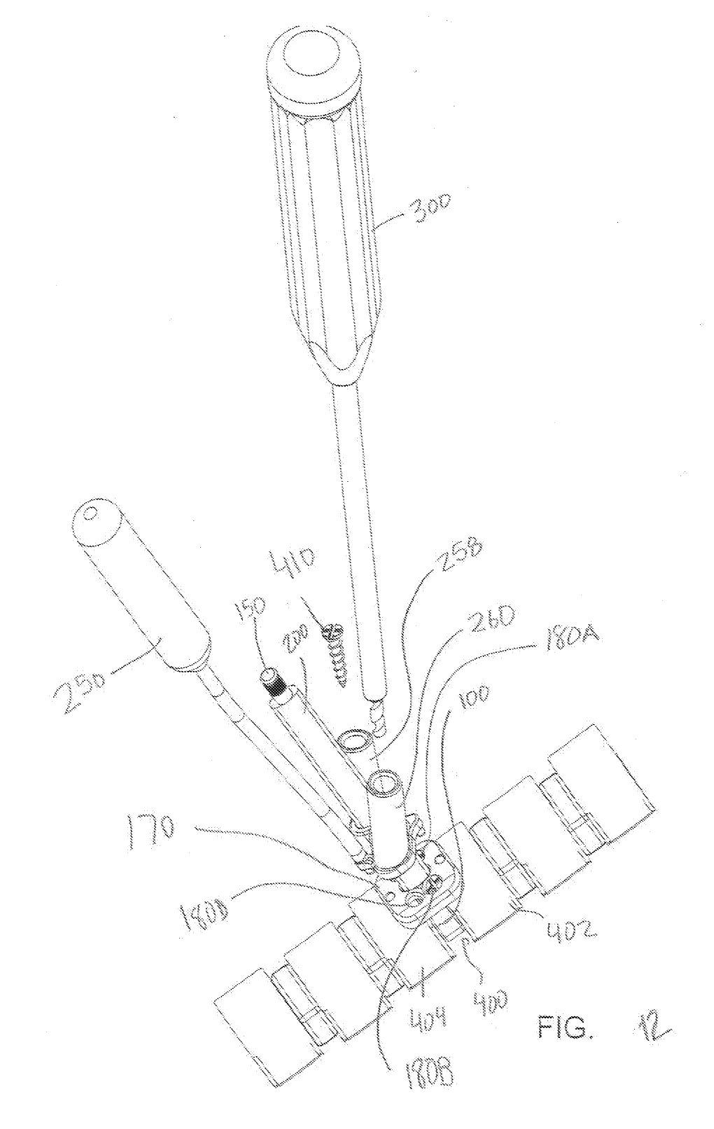

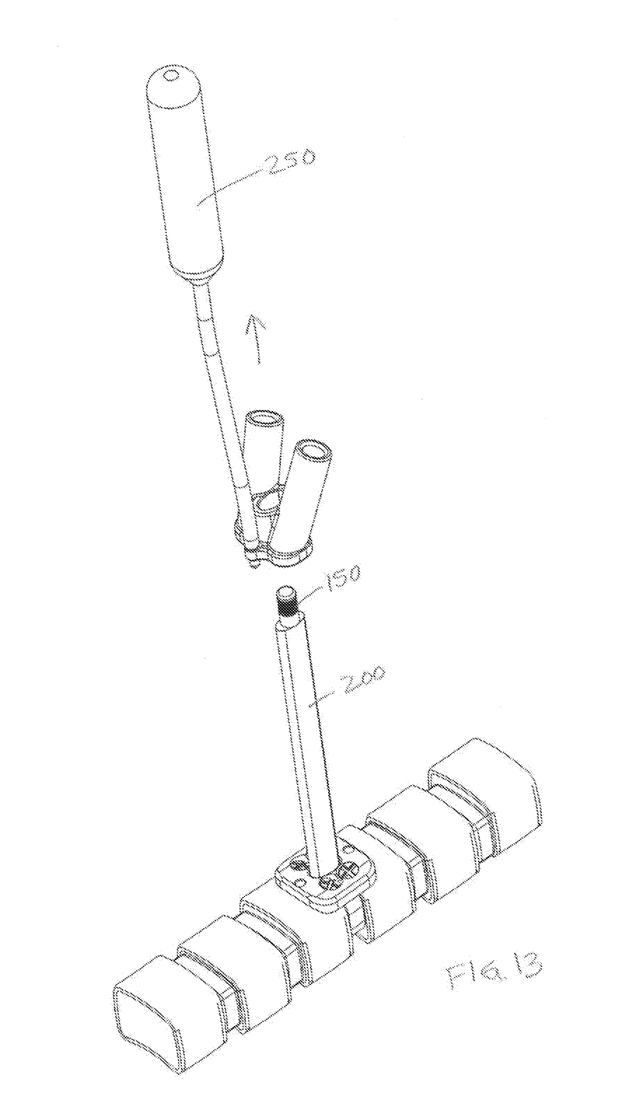

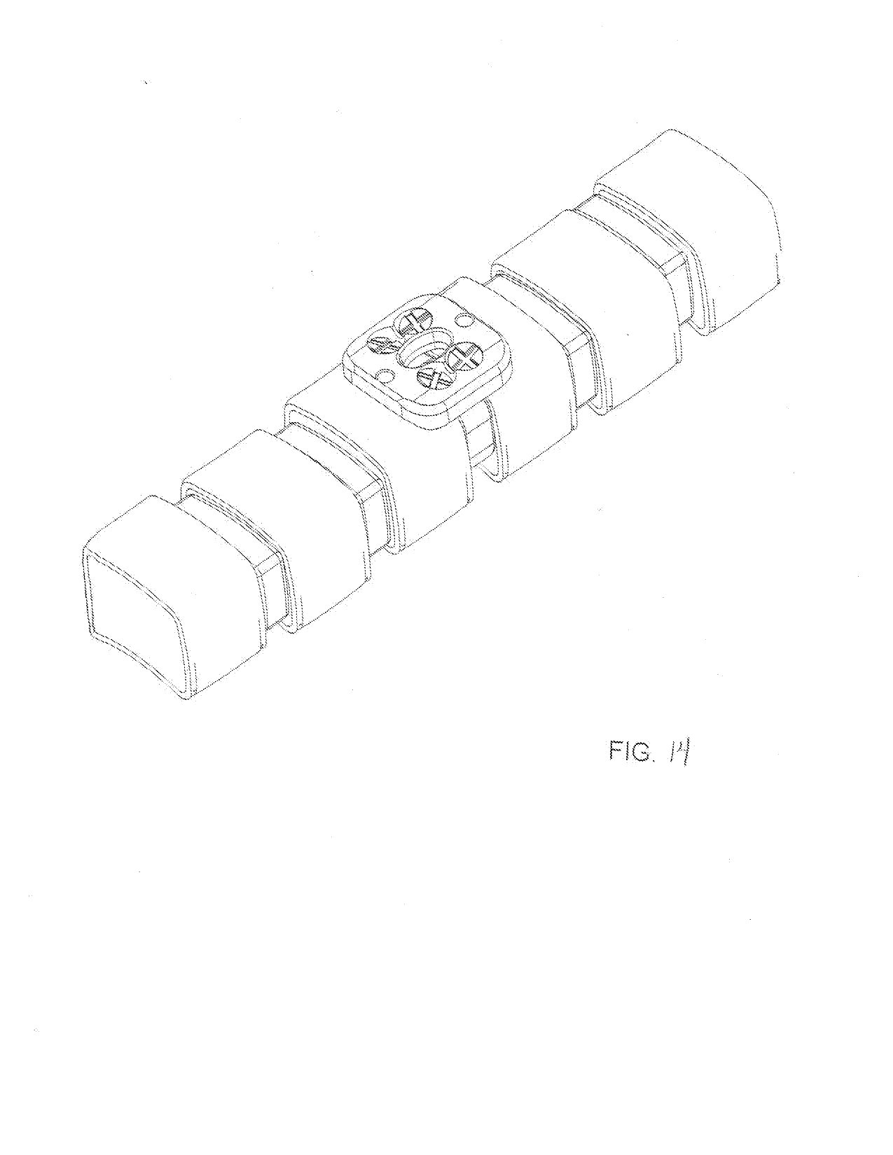

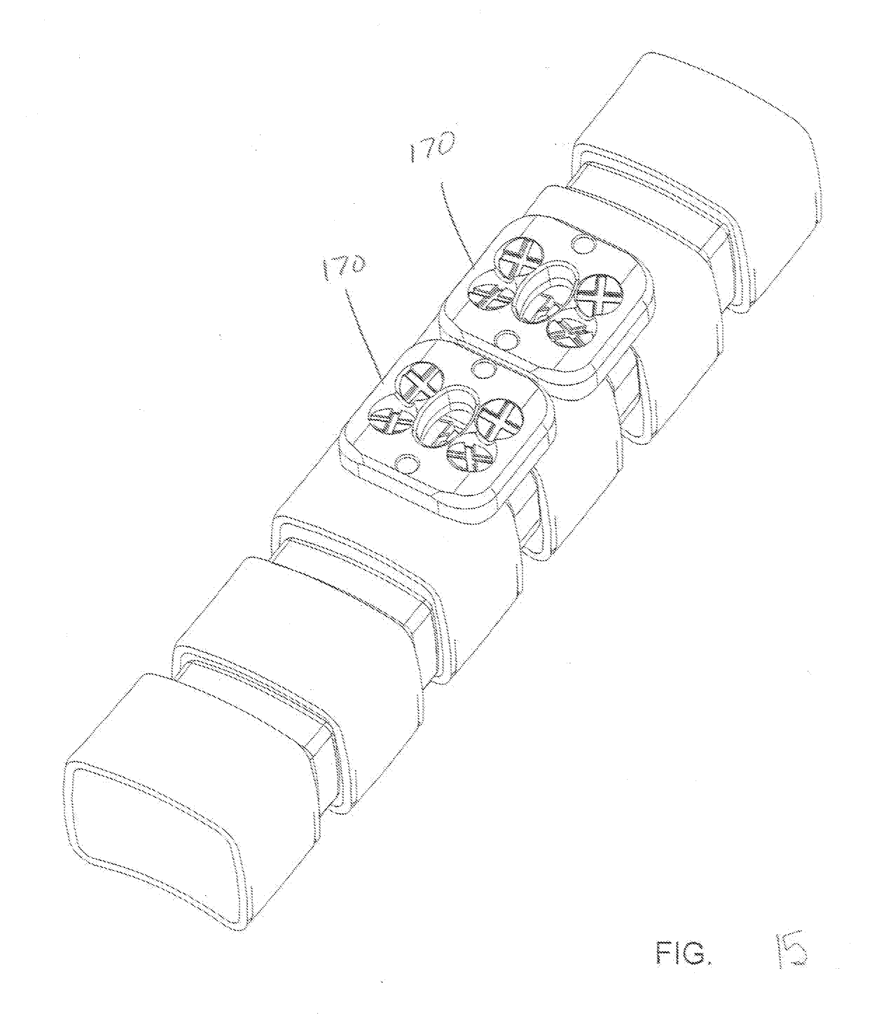

As seen in FIG. 12, once the fasteners 410 have been installed into the first set of holes 180A, 180B in the first vertebra 402, the drill guide 250 may be removed from the guide member 200, reoriented by 180 degrees, and then reinstalled onto the guide member 200. The drill 300 may then be utilized to drill pilot holes in the vertebra 404. Again fasteners 410 may be utilized to secure the interbody plate 170 to the vertebra 404 through the second set of holes 180C, 180D in the plate 170. With the interbody plate 170 secured, the drill guide 250 and the elongated guide member 200 may be removed as shown in FIGS. 13 and 14. In particular, to remove the elongated guide member 200, the threads 158 on the distal end 154 of the guide member 200 may be unscrewed from the threaded bore 112 of the interbody cage 100. As shown in FIG. 15, multiple interbody plates 170 may be utilized to fuse several levels of vertebrae of a patient.

It will be appreciated that although the engagement that removably secures the guide member 200 to the interbody cage 100 has been described herein as a threaded engagement between the holding rod 150 and the threaded bore 112, that any engagement that removably secures the guide member 200 to the interbody cage 100 falls within the scope of the present disclosure. Further, it will be appreciated that the grooves in the superior surface 104 and the inferior surface 105 of the body member 102 of the interbody cage 100 allow the fasteners utilized to secure the interbody plate 170 to pass within the grooves.

It will be appreciated that it is a distinct advantage of the present disclosure to accurately position the interbody plate 170 above a vertebral gap. This accurate positioning of the plate 170 may allow the length of the interbody plate 170 to be kept to a minimum, which may allow the use of multiple plates 170 at multiple adjacent levels in the spine as shown in FIG. 15. This feature may have biomechanical advantages and improve fusion rates over single long plates. It also means that where access is limited, such as in minimally invasive procedures, the more easily small plates can be selected and positioned without fear the screws will damage or move a cage installed between the vertebral space.

Further, it will be appreciated that using the plate 170 would mean that the bone fasteners may cut into the cage 100 without the grooves in the superior and inferior surfaces of the cage 100 (see FIG. 1). In an embodiment, the grooves are positioned to align with the shortest plates possible for any given size of cage so that even with a very short plate, the fasteners will not cut the cage.

In an embodiment, the plate 170 may be utilized as a buttress plate that is only secured to a single vertebra and whose sole function is prevent back out of a cage. It will be appreciated that this may be useful where a second stage adjustment of the relative position of the vertebrae is desired and would be limited by having fasteners installed into both vertebrae. Such a buttress plate may have one or two screws, but again correct alignment and ease of placement would be helpful using the concepts of the present invention.

Those having ordinary skill in the relevant art will appreciate the advantages provided by the features of the present disclosure. For example, it is a feature of the present disclosure to provide a spinal plate selection and positioning system. Another feature of the present disclosure is to provide a guide member that removably attaches to an interbody cage, the guide member aligning and positioning an interbody plate and drill guide. It is a further feature of the present disclosure, in accordance with one aspect thereof, to provide an interbody cage with grooves in its superior and inferior surfaces for allowing passage of fasteners utilized to secure an interbody plate.

In the foregoing Detailed Description, various features of the present disclosure are grouped together in a single embodiment for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description of the Disclosure by this reference, with each claim standing on its own as a separate embodiment of the present disclosure.

It is to be understood that the above-described arrangements are only illustrative of the application of the principles of the present disclosure. Numerous modifications and alternative arrangements may be devised by those skilled in the art without departing from the spirit and scope of the present disclosure and the appended claims are intended to cover such modifications and arrangements. Thus, while the present disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A method for stabilizing a spine, the spine having a first vertebra and a second vertebra, the first vertebra and the second vertebra being adjacent and defining a disc space between them, said method comprising:
installing an interbody cage in the disc space between the first vertebra and the second vertebra;
aligning and positioning an interbody plate over the disc space using an elongated guide member extending from, and removably secured to, the interbody cage located within the disc space;
securing the interbody plate to at least one of the first vertebra and the second vertebra; and
removing the elongated guide member from the interbody cage.

2. The method of claim 1, further comprising aligning and positioning a drill guide using the elongated guide member extending from, and removably secured to, the interbody cage.

3. The method of claim 2, further comprising drilling pilot holes in the first vertebra and the second vertebra using the drill guide.

4. The method of claim 2, further comprising aligning and positioning the drill guide using the elongated guide member such that the interbody plate is interposed between the interbody cage and the drill guide.

5. The method of claim 4, further comprising drilling pilot holes in the first vertebra and the second vertebra with the interbody plate interposed between the drill guide and the interbody cage.

6. The method of claim 5, further comprising securing the interbody plate to the first vertebra and the second vertebra using the drill guide to guide fasteners into the pilot holes.

7. The method of claim 5, wherein the pilot holes comprise a first set of pilot holes and a second set of pilot holes, and the method further comprises drilling the first set of pilot holes with the drill guide in a first orientation with respect to the elongated guide member and drilling the second set of pilot holes with the drill guide in a second orientation with respect to the elongated guide member, wherein the first orientation and the second orientation are different.

8. The method of claim 7, further comprising reorientating the drill guide 180 degrees between the first orientation and the second orientation.

9. The method of claim 1, further comprising securing the elongated guide member to the interbody cage using a holding rod.

10. The method of claim 9, further comprising inserting a threaded end of the holding rod into a cannulation in the elongated guide member and threading the threaded end of the holding rod into a threaded bore in the interbody cage.

11. The method of claim 10, further comprising locking the elongated guide member to the interbody cage such that the elongated guide member is prevented from rotating around a longitudinal axis of the elongated guide member.

12. The method of claim 11, further comprising inserting a key member extending from the elongated guide member into a key slot in the interbody cage.

13. The method of claim 12, wherein the elongated guide member comprises a noncircular cross section in a plane perpendicular to its longitudinal axis.

14. The method of claim 13, wherein the elongated guide member comprises an oval cross section.

15. The method of claim 13, wherein the interbody plate comprises a guide hole complementary in shape to the noncircular cross section of the elongated guide member, and the method further comprises installing a proximal end of the elongated guide member into the guide hole of the interbody plate and sliding the interbody plate down the elongated guide member into position over the disc space.

16. The method of claim 15, wherein the drill guide comprises a guide hole complementary in shape to the noncircular cross section of the elongated guide member, and the method further comprises installing the proximal end of the elongated guide member into the guide hole of the drill guide and sliding the drill guide into position over the interbody plate.

17. A system for fusing a first vertebra and a second vertebra of a spinal column, the first vertebra and second vertebra defining a disc space, said system comprising:
an interbody cage having a proximal end and a distal end, the interbody cage configured and dimensioned to fit within the disc space;
an elongated guide member removably attachable to the interbody cage, the elongated guide member extending along a longitudinal axis from a proximal end to a distal end, the elongated guide member having an outer surface; and
an interbody plate having an inner surface defining a guide hole, said guide hole configured and dimensioned to receive the elongated guide member to thereby align and position said interbody plate over the disc space;
wherein the inner surface of the interbody plate engages the outer surface of the elongated guide member to prevent the interbody plate from rotating about the longitudinal axis of the elongated guide member.

18. The system of claim 17, wherein the interbody cage comprises:
a body member, the body member having a superior surface and an inferior surface;
the body member further having a sidewall extending between the superior surface and the inferior surface, the sidewall circumscribing the body member;
a first set of grooves formed in the superior surface, the first set of grooves extending from the proximal end towards the distal end; and
a second set of grooves formed in the inferior surface, the second set of grooves extending from the proximal end towards the distal end.

19. The system of claim 17, wherein the interbody plate comprises a plurality of holes for receiving fasteners such that the interbody plate is securable to the first vertebra and the second vertebra.

20. The system of claim 19, wherein the holes comprise a first set of holes and a second set of holes.

21. The system of claim 20, wherein one of the first set of holes is disposed on either side of the guide hole and one of the second set of holes is disposed on either side of the guide hole.

22. The system of claim 21, wherein each of the first set of holes and each of the second set of holes comprises an axis, wherein the axis of each of the first set of holes and the second set of holes forms an acute angle with a plane containing an axis of the guide hole.

23. The system of claim 17, wherein the guide hole is non-circular.

24. The system of claim 17, wherein the proximal end of the interbody cage further comprises a threaded bore.

25. The system of claim 24, further comprising a holding rod having a threaded end, said threaded end configured and dimensioned to engage the threaded bore of the interbody cage.

26. The system of claim 25, wherein the elongated guide member comprises a cannulation along its longitudinal axis, and the holding rod is configured and dimensioned to pass through the cannulation such that the threaded end of the holding rod can be installed in the threaded bore of the interbody cage.

27. The system of claim 26, wherein the proximal end of the interbody cage comprises a key slot, and wherein a distal end of the elongated guide member comprises a key, and wherein said key of the elongated guide member is removably installable in the key slot of the interbody cage such that the elongated guide member is prevented from rotating about its longitudinal axis.

28. The system of claim 17, further comprising a key and a key slot to prevent the elongated guide member from rotating about its longitudinal axis.

29. A system for joining a first vertebra and a second vertebra of a spinal column, the first vertebra and second vertebra defining a disc space, said system comprising:
an interbody cage having a proximal end and a distal end, the interbody cage configured and dimensioned to fit within the disc space;
an elongated guide member removably attachable to the interbody cage, the elongated guide member having an outer surface and extending from a proximal end to a distal end along a longitudinal axis; and
a drill guide having an inner surface defining a guide hole, said guide hole configured and dimensioned to receive the elongated guide member such that said drill guide can slide down the elongated guide member into position over the interbody cage;
wherein the inner surface of the drill guide engages the outer surface of the elongated guide member to prevent the drill guide from rotating about the longitudinal axis of the elongated guide member.

30. The system of claim 29, wherein the interbody cage comprises:
a body member, the body member having a superior surface and an inferior surface;
the body member further having a sidewall extending between the superior surface and the inferior surface, the sidewall circumscribing the body member;
a first set of grooves formed in the superior surface, the first set of grooves extending from the proximal end towards the distal end; and
a second set of grooves formed in the inferior surface, the second set of grooves extending from the proximal end towards the distal end.

31. The system of claim 29, wherein the drill guide comprises a plurality of bit guide tubes for receiving a drill bit.

32. The system of claim 31, wherein the plurality of bit guide tubes are angled.

33. The system of claim 32, further comprising an interbody plate, the interbody plate having a plurality of holes in alignable with the bit guide tubes.

34. The system of claim 29, wherein the guide hole is non-circular.

35. The system of claim 34, wherein the guide hole is oval.

36. The system of claim 29, wherein the proximal end of the interbody cage further comprises a threaded bore.

37. The system of claim 36, further comprising a holding rod having a threaded end, said threaded end configured and dimensioned to engage the threaded bore of the interbody cage.

38. The system of claim 37, wherein the elongated guide member comprises a cannulation along its longitudinal axis, and the holding rod is configured and dimensioned to pass through the cannulation such that the threaded end of the guide rod can be installed in the threaded bore of the interbody cage.

39. The system of claim 38, wherein the proximal end of the interbody cage comprises a key slot, and wherein a distal end of the elongated guide member comprises a key, and wherein said key of the elongated guide member is removably installable in the key slot of the interbody cage such that the elongated guide member is prevented from rotating about its longitudinal axis.

40. The system of claim 29, further comprising a key and a key slot to prevent the elongated guide member from rotating about its longitudinal axis.

41. An interspinous device for use in a spinal fixation surgery, said device comprising:
an interbody cage, the interbody cage having a superior surface and an inferior surface;
the interbody cage further having a sidewall extending between the superior surface and the inferior surface, the sidewall circumscribing the interbody cage, the sidewall defining a distal end and a proximal end of the interbody cage;
a first set of grooves formed in the superior surface, the first set of grooves extending from the proximal end towards the distal end; and
a second set of grooves disposed on the inferior surface, the second set of grooves extending from the proximal end towards the distal end.

42. The interspinous device of claim 41, further comprising a threaded bore extending into the proximal end of the interbody cage.

43. The interspinous device of claim 42, further comprising a key slot in the proximal end of the interbody cage.

44. The interspinous device of claim 42, wherein the key slot extends across an opening of the threaded bore in the proximal end of the interbody cage.

45. A spinal fixation device comprising:
an interbody plate having a proximal end and a distal end;
the interbody plate having an inner sidewall defining a guide hole, the guide hole being non-circular; and
the interbody plate having a first set of holes and a second set of holes.

46. The spinal fixation device of claim 45, wherein the guide hole is centrally located on the interbody plate.

47. The spinal fixation device of claim 46, wherein the guide hole is oval.

48. The spinal fixation device of claim 45, wherein one of the first set of holes is disposed on either side of the guide hole and one of the second set of holes is disposed on either side of the guide hole.

49. The spinal fixation device of claim 48, and wherein each of the first set of holes and each of the second set of holes are splayed.

50. The spinal fixation device of claim 45, further comprising an elongated guide member configured and adapted to be inserted into the guide hole.

51. The spinal fixation device of claim 45, wherein the elongated guide member comprises an outer surface, and wherein the inner sidewall of the interbody plate engages the outer surface of the elongated guide member to prevent the interbody plate from rotating about a longitudinal axis of the elongated guide member.

52. An interspinous system comprising:
an interbody cage having a proximal end and a distal end, the interbody cage configured and dimensioned to fit within a disc space between a first vertebra and a second vertebra;
a guide member removably attachable to the interbody cage, the guide member extending along a longitudinal axis from a proximal end to a distal end, the elongated guide member having an outer surface and an elongated cross section; and
an interbody plate having an inner surface defining a guide hole, said guide hole configured and dimensioned to receive the guide member to thereby align and center said interbody plate with respect to the interbody cage;
wherein the inner surface of the interbody plate engages the outer surface of the guide member to prevent the interbody plate from rotating about the longitudinal axis of the elongated guide member.

53. A system for joining a first vertebra and a second vertebra of a spinal column, the first vertebra and second vertebra defining a disc space, said system comprising:
an interbody cage, the interbody cage configured and dimensioned to fit within the disc space, the interbody cage having a superior surface and an inferior surface, the interbody cage having a sidewall extending between the superior surface and the inferior surface, the sidewall circumscribing the interbody cage, the sidewall defining a proximal end and a distal end of the interbody cage, the superior surface having a first set of grooves formed therein, the first set of grooves extending from the proximal end towards the distal end, the inferior surface having a second set of grooves formed therein, the second set of grooves extending from the proximal end towards the distal end, a threaded bore extending into the proximal end of the sidewall, and a key slot extending over the threaded bore;
an elongated guide member removably attachable to the interbody cage, the elongated guide member having a proximal end, a distal end, an outer surface and a longitudinal axis, the elongated guide member having a cannulation extending along the longitudinal axis from the proximal end to the distal end, the elongated guide member have a key member extending from its distal end, wherein said key member is configured and adapted to mate with said key slot in the interbody cage, the elongated guide member having a non-circular cross-section in a plane perpendicular to its longitudinal axis;
a holding rod, the holding rod having a threaded end, said threaded end configured and dimensioned to engage the threaded bore of the interbody cage, the holding rod configured and dimensioned to be inserted into the cannulation of the elongated guide member such that the holding rod secures the elongated guide member to the interbody cage;
an interbody plate having a proximal end and a distal end, the interbody plate having an inner sidewall defining a guide hole, the guide hole extending from the proximal end to the distal end, the interbody plate having a first set of holes and a second set of holes, wherein the first set of holes are splayed in a first direction, wherein the second set of holes are splayed in a second direction, wherein the guide hole is centrally located on the interbody plate, wherein the guide hole is non-circular, wherein one of the first set of holes is disposed on either side of the guide hole and one of the second set of holes is disposed on either side of the guide hole, wherein the elongated guide member is configured and adapted to be inserted into the guide hole, and wherein the inner sidewall of the interbody plate engages the outer surface of the elongated guide member to prevent the interbody plate from rotating about the longitudinal axis of the elongated guide member; and
a drill guide, the drill guide having an inner surface defining a guide hole, said guide hole configured and dimensioned to receive the elongated guide member such that said drill guide can slide down the elongated guide member into position over the disc space, wherein the inner surface of the drill guide engages the outer surface of the elongated guide member to prevent the drill guide from rotating about the longitudinal axis of the elongated guide member, the drill guide comprising a plurality of bit guide tubes for receiving a drill bit.
